(19) United States
(12) Plant Patent Application Publication
(10) Pub. No.: US 2026/0314510 P1
(43) Pub. Date: Oct. 8, 2026

(54) Garden Rose Plant Named 'VLR13015'

(50) ;

(71) Applicants: VINELAND RESEARCH AND INNOVATION CENTRE, Vineland Station (CA); Canadian Nursery Landscape Association, Milton (CA)

(72) Inventor: Parminderjit SANDHU, Vineland Station (CA)

(73) Assignees: VINELAND RESEARCH AND INNOVATION CENTRE, Vineland Station, ON (CA); Canadian Nursery Landscape Association, Milton, ON (CA)

(21) Appl. No.: 19/435,912

(22) Filed: Dec. 30, 2025

(30) Foreign Application Priority Data
Apr. 7, 2025 (CA) 25-11988

(51) Int. Cl. A01H 6/74; A01H 5/02

(52) U.S. Cl. CPC A01H 6/749; A01H 5/02
USPC PLT/105

(57) ABSTRACT

A new and distinct variety of garden rose plant, referred to by its cultivar name, ‘VLR13015’, is described. The new plant variety forms in abundance on a substantially continuous basis very full orange-colored blossoms. The growth habit is intermediate shrub. Semi-glossy, dark green foliage is formed. Additionally, the new plant variety is particularly well suited for growing as distinctive ornamentation in the landscape.

### CROSS REFERENCE TO RELATED APPLICATIONS

This application claims priority to Canadian Plant Breeders' Rights Application No. 25-11988, which was filed on Apr. 7, 2025, the contents of which are hereby incorporated by reference for all purposes.

Latin name of genus and species of plant claimed: Rosa hybrida.

Variety denomination: ‘VLR13015’.

### BACKGROUND OF THE INVENTION

The new variety of garden rose plant of the present invention was created by controlled breeding in 2013 in Vineland Station, Ontario, Canada by artificial pollination wherein two parents were crossed which previously had been studied in the hope that they would contribute the desired characteristics. The female parent (i.e., the seed parent) of the new plant variety was ‘20ALFR05’ (not patented). The male parent (i.e., the pollen parent) of the new plant variety was ‘27YSPO412’ (not patented).

The parentage of the new plant variety can be summarized as follows:
‘20ALFR05’×‘27YSPO412’

The seeds resulting from the above pollination were sown and small plants were obtained which were physically and biologically different from each other. Selective study resulted in the identification of a single plant of the new variety.

The new plant variety has been found to undergo asexual propagation at Vineland Station, Ontario, Canada by a number of routes such as vegetative cuttings. Asexual propagation techniques in Vineland Station, Ontario, Canada, such as vegetative cuttings, have shown that the characteristics of the new plant variety are homogeneous, stable, and strictly transmissible by such asexual propagation from one generation to another. Accordingly, the new plant variety undergoes asexual propagation in a true-to-type manner.

### SUMMARY OF THE INVENTION

It was found that the new variety of garden rose plant of the present invention possesses the following combination of characteristics:
(a) abundantly and substantially continuously forming very full orange-colored blossoms,
(b) exhibiting an intermediate shrub growth habit, and
(c) forming semi-glossy, dark green foliage.

The new plant variety well meets the needs of the horticultural industry. It can be grown to advantage as distinctive ornamentation in parks, gardens, public areas, and residential landscapes. Accordingly, it is particularly well suited for growing in the landscape.

The new plant variety can be readily distinguished from its ancestors. Moreover, the new plant variety can be readily distinguished from non-parental related similar varieties. For example, the new plant variety displays very full orange-colored flowers and forms dark green foliage, whereas the ‘Meivildo’ variety (U.S. Plant Pat. No. 6,895) displays wider violet-colored flowers and forms green foliage with larger sized leaflets compared to the new plant variety. Additionally, the new plant variety has improved winter hardiness up to USDA Zone 4b compared to the ‘Meivildo’ variety which is cold hardy up to USDA Zone 5b.

The new plant variety has been named the ‘VLR13015’ variety.

### BRIEF DESCRIPTION OF THE PHOTOGRAPHS

The accompanying photographs show, as nearly true as it is reasonably possible to make the same in a color illustration of this character, typical specimens of the new plant variety and blossoms of the new plant variety. The illustrated rose plant of the new variety was grown in Vineland Station, Ontario, Canada.

FIG. 1—illustrates a side-by-side view of the flower buds of ‘VLR13015’, left, and ‘Meivildo’, right.

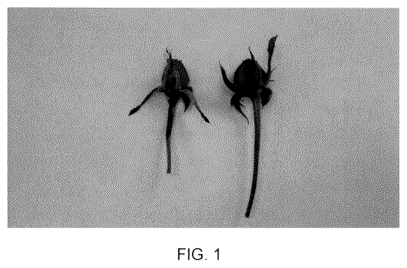

FIG. 2—illustrates a side-by-side top view of opened flowers of ‘VLR13015’, left, and ‘Meivildo’, right.

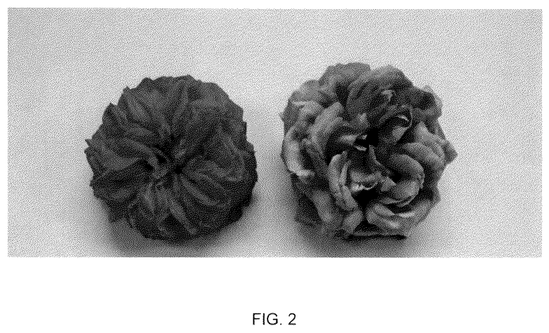

FIG. 3—illustrates a side-by-side view of the upper side of the leaves of ‘VLR13015’, left, and ‘Meivildo’, right.

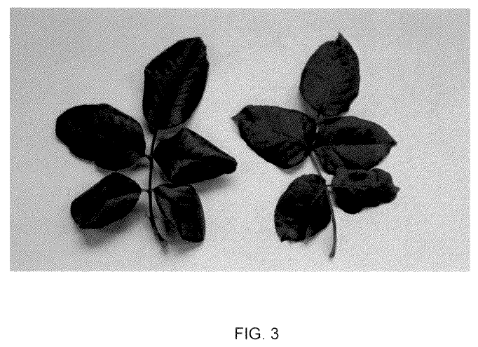

### DETAILED BOTANICAL DESCRIPTION

The chart used in the identification of colors is that of The Royal Horticultural Society (The R.H.S. Colour Chart, 2007 edition). The terminology which precedes reference to the chart has been added to indicate the corresponding color in more common terms. The R.H.S. Colour Chart designation used herein represents the closest color observed on the majority of the specified botanical features. The description is based on a two-year-old specimen of the new variety, observed during September 2025, while growing in a three-gallon container in Vineland Station, Ontario, Canada.
Botanical classification: Rosa hybrida cultivar ‘VLR13015’.
Commercial classification: Garden Rose
Subclass: Shrub
Plant:
   Habit.—Intermediate shrub.
   Height.—Approximately 65.2 cm on average during second flush.
   Width.—Approximately 81 cm on average.
Branches:
   Stem color.—Old wood: 137C. — young stems: 138B.
   Length.—Main stems: approximately 56 cm on average. — secondary stems: approximately 51 cm on average.
   Diameter.—Main stems: approximately 3.0 cm on average. — secondary stems: approximately 0.8 cm on average.
   Internode length.—Main stems: approximately 5.1 cm on average. — secondary stems: approximately 3.4 cm on average.
   Texture.—Main stems: woody with prickles. — secondary stems: woody with prickles.
   Anthocyanin coloration of young shoots.—very weak, 152D.
   Prickles.—Amount: medium, 4 per internode. — young prickles: length is approximately 5.0 mm on average; width is approximately 2.5 mm on average at point of attachment; and color is 144B. — old prickles: length is approximately 1.1 cm on average; width is approximately 9 mm on average at point of attachment; and color is 165C.
   Flowering shoots.—Number of flowering laterals: many, typically 4. — number of flowers (only shoots with no flowering laterals): very few, typically 3. — number of flowers (only shoots with flowering laterals): medium, typically 5.
Leaflets:
   Number.—Mostly five.
   Shape of terminal leaflet.—Narrow elliptic; apex is acute; and base is obtuse.
   Shape of lateral leaflet.—Elliptic; apex is acute; and base is obtuse.
   Arrangement.—Odd-pinnate.
   Margin.—Serrate.
   Texture.—Upper surface: glabrous. — under surface: scabrous.
   Venation pattern.—Reticulate.
   Undulation.—Weak.
   Glossiness of upper side.—Medium.
   Glossiness of lower side.—Dull.
   Intensity of green color on upper side.—Dark.
   Intensity of green color on lower side.—Light green.
   Anthocyanin coloration of newly opened leaflets.—Weak, 152D.
   Size.—Terminal leaflet: length is approximately 6.9 cm on average; width is approximately 4.3 cm on average. — lateral leaflets: length is approximately 5.8 cm on average; width is approximately 3.6 cm on average. — leaf: length is approximately 11.7 cm on average; width is approximately 11.0 cm on average.
   Petiole.—Texture: smooth. — length: approximately 2.3 cm on average. — diameter: approximately 2.0 mm on average. — color: 137D.
   Rachis.—Texture: bit rough with a few thorns. — length: approximately 4.2 cm on average. — diameter: approximately 1.25 mm on average. — color: 138B.
   Petiolules.—Texture: smooth. — length: approximately 3 mm on average. — diameter: approximately 1.0 mm on average. — color: 138C.
Foliage:
   Young foliage.—Upper surface color: 158D with N138B venation. — under surface color: 152C with 139C venation.
   Old foliage.—Upper surface color: N137B with 139A venation. — under surface color: 147B with 137D venation.
   Anthocyanin coloration of mature leaves.—Absent.
Inflorescence:
   Number of flowers.—About 16 blooms on average on a plant at once.
   Number of blossoms per stem or in a cluster.—Typically 4 blooms per stem on average.
   Peduncle.—Color: 138A. — diameter: approximately 2.5 mm on average. — length: approximately 5.1 cm on average. — surface texture: smooth.
   Sepals.—Upper surface color and texture: fine soft hairs, 138C. — under surface color and texture: smooth, 138B. — size: length is approximately 3.4 cm on average; width is approximately 8 mm on average. — margin: smooth. — undulation: absent. — shape: urceolate. — apex: pointed. — extensions: absent or very weak.
   Buds.—Shape: medium ovate. — color (just before the separation of the sepals): Green Group 138A.
   Flower form.—Double. — shape: round. — profile: upper part is flattened convex; lower part is concave. — diameter: approximately 7.2 cm on average. — depth: approximately 2.7 cm on average. — duration: on the plant approximately 7 days. — color group: orange blend. — color of the center when viewed from above: orange, 41C. — petals reflex one by one: no.
   Fragrance.—Absent or weak.
   Petal.—Number: approximately 119 on average. — length: approximately 2.8 cm on average. — width: approximately 2.5 cm on average. — overall shape: rounded. — margin: wavy. — reflexing of margin: weak. — undulation: absent or very weak. — apex shape: aristate. — base shape: narrow. — texture (both surfaces): smooth. — incisions: absent or very weak. — density of petals: dense. — number of colors on inner side (basal spot excluded): one. — intensity of color: even. — main color on the inner side: Red Group 44D. — basal spot on inner side: small, with a color of light yellow, 8D. — main color on the outer side: Red Group 43D.
   Stamen.—Number: typically 24. — anthers: color is 15D; length is approximately 2.5 mm on average; width is approximately 1.0 mm on average; and shape is rectangular. — filaments: length is approximately 2.5 mm on average, diameter is approximately 0.3 mm on average, and color of filament of outer stamen is orange 31D.
   Pistils.—Number: typically 92. — style: length is approximately 3.0 mm on average, diameter is approximately 0.25 mm on average, and color is 39A. — stigma: length is approximately 0.5 mm on average, width is approximately 0.5 mm on average; shape is round; color is 33D.
   Receptacle.—Diameter: approximately 9 mm on average. — depth: approximately 8 mm on average. — color: 138B.
   Pollen.—Amount: scarce. — color: 16C.
   Seed vessel.—Size at petal fall: medium, length is approximately 8 mm on average, diameter is approximately 8 mm on average.
   Hip.—Shape in longitudinal section: pitcher-shaped. — color at mature stage: orange 30D.
Development:
   Vegetation.—Semi-glossy, dark green.
   Blossoming.—Typically June to October.
   Resistance to diseases.—Resistant to black spot (Diplocarpon rosae).
   Hardiness.—Hardy to USDA Zone 4b.

The new ‘VLR13015’ variety has not been observed under all possible environmental conditions to date. Accordingly, it is possible that the phenotypic expression may vary somewhat with changes in light intensity and duration, cultural practices, and other environmental conditions.

I claim:

1. A new and distinct variety of garden rose plant named ‘VLR13015’ characterized by the following combination of characteristics:
(a) abundantly and substantially continuously forming very full orange-colored blossoms,
(b) exhibiting a shrubbyan intermediate shrub growth habit, and
(c) forming semi-glossy, dark green foliage;
substantially as herein shown and described.
